(19) United States
(12) Patent Application Publication
(10) Pub. No.: US 2026/0192041 A1
(43) Pub. Date: Jul. 9, 2026

(54) BELOW PUMP SEGMENT AIR IN LINE CAPTURE

(71) Applicant: CareFusion 303, Inc., San Diego, CA (US)

(72) Inventors: Jason Andrew WINE, Brea, CA (US); Eugene E. MASON, La Habra Heights, CA (US)

(21) Appl. No.: 19/012,037

(22) Filed: Jan. 7, 2025

(51) Int. Cl. A61M 5/142; A61M 5/168

(52) U.S. Cl. CPC A61M 5/142; A61M 5/16831; A61M 2205/0233; A61M 2205/18; A61M 2205/3327; A61M 2205/3331; A61M 2205/3375; A61M 2205/50

(57) ABSTRACT

Devices for capturing air in a fluid pathway are provided. The device includes IV tubing configured to be disposed within an air-in-line sensor and a flow cannula disposed within the IV tubing. The flow cannula includes a center lumen disposed axially along the flow cannula, wherein the flow cannula is configured to direct an incoming fluid flow from a pump segment through the center lumen into an air capture device and to direct processed fluid received back from the air capture device through the flow cannula externally to the center lumen and internally to the IV tubing. Methods for capturing air in a fluid pathway and flow cannulas for use within IV tubing are also provided.

### FIELD OF THE INVENTION

The present disclosure generally relates to fluid pumps, and in particular to providing fluid path access for air capturing devices.

### BACKGROUND

Medical treatments often include the infusion of a medical fluid such as a saline solution or a liquid medication to patients using an intravenous (IV) catheter that is connected though an IV set having an arrangement of flexible tubing and fittings to a source of fluid (e.g., IV bag or a medication container) and infused to the patient by an infusion pump. Typical methods are used to capture and separate air from an IV fluid administration path, such as a dedicated device that can be installed in the fluid line.

A challenge arises when these methods are applied to a large volume pump system. Air bubbles may form within the pump segment during use of the large volume pump. These air bubbles may be the source of many air-in-line sensor alarms. If the air capturing device is placed above the large volume pump, then the bubbles generated within the pump segment will not be captured by the air capturing device, thus resulting in air ingress risk to the patient and nuisance air-in-line alarms for clinicians. On the other hand, if the air capturing device is placed below the large volume pump, then the bubbles generated within the pump segment will trigger the air-in-line alarm of the pump prior to being captured. Here, the air ingress risk to the patient is reduced, but the nuisance of air-in-line alarms for clinicians is not avoided.

### SUMMARY

One or more embodiments of the present disclosure are directed to a device for capturing air in a fluid pathway, the device including intravenous (IV) tubing configured to be disposed within an air-in-line sensor and a flow cannula disposed within the IV tubing, the flow cannula comprising a center lumen disposed axially along the flow cannula, wherein the flow cannula is configured to direct an incoming fluid flow from a pump segment through the center lumen and into an air capture device, and wherein the flow cannula is configured to direct processed fluid received back from the air capture device through the flow cannula externally to the center lumen and internally to the IV tubing.

One or more embodiments of the present disclosure are directed to a method for capturing air in a fluid pathway, the method including placing intravenous (IV) tubing comprising a flow cannula within an air-in-line sensor of an infusion pump; positioning the flow cannula between a transmitter and a receiver of the air-in-line sensor; directing fluid flow from the infusion pump through a center lumen of the flow cannula having a center lumen wall configured to reflect waves from the transmitter from passing through the center lumen; directing fluid flow from the center lumen into an air capture device; and directing fluid flow from the air capture device back into the flow cannula to fill one or more channels around the center lumen wall with fluid.

One or more embodiments of the present disclosure are directed to a flow cannula for use within intravenous (IV) tubing, the flow cannula including an axially disposed center lumen wall defining a center lumen; a first channel wall extending radially from the center lumen wall; a second channel wall extending radially from the center lumen wall and on an opposite side of the center lumen wall from the first channel wall; a first flow channel disposed between a first side of the first channel wall, a first side of the second channel wall and a first portion of the center lumen wall; a second flow channel disposed between a second side of the first channel wall, a second side of the second channel wall and a second portion of the center lumen wall; and a flow hole disposed in one of the first channel wall and the second channel wall, the flow hole fluidly coupling the first flow channel and the second flow channel.

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1A depicts a perspective view of an example patient care system having a pump control unit and four fluid infusion pumps, each of which is connected to a respective fluid supply for pumping the contents of the fluid supply to a patient, according to aspects of the disclosure.

FIG. 1B is a perspective view of a pump segment within an infusion pump, according to aspects of the disclosure.

FIG. 2A is a front view of an IV set having a flow path device, according to aspects of the disclosure.

FIG. 2B is a cross-sectional partial view of the IV set of FIG. 2A, according to aspects of the disclosure.

FIG. 3A is a perspective partial view of an IV set having a flow path device, according to aspects of the disclosure.

FIG. 3B is a cross-sectional side partial view of the IV set of FIG. 3A, according to aspects of the disclosure.

FIG. 3C is a cross-sectional top view of the IV set of FIG. 3B, according to aspects of the disclosure.

### DETAILED DESCRIPTION

In the following detailed description, numerous specific details are set forth to provide a full understanding of the subject technology. It should be understood that the subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of fluid connectors, such embodiments can be used in other systems that benefit from creating sterile connections. Furthermore, various applications of such embodiments and modifications thereto, which may occur to those who are skilled in the art, are also encompassed by the general concepts described herein.

In aspects of the disclosure, a means or device is provided to provide a fluid path access for an air capturing device that is below a pump segment of an infusion pump and above an air-in-line sensor of the infusion pump.

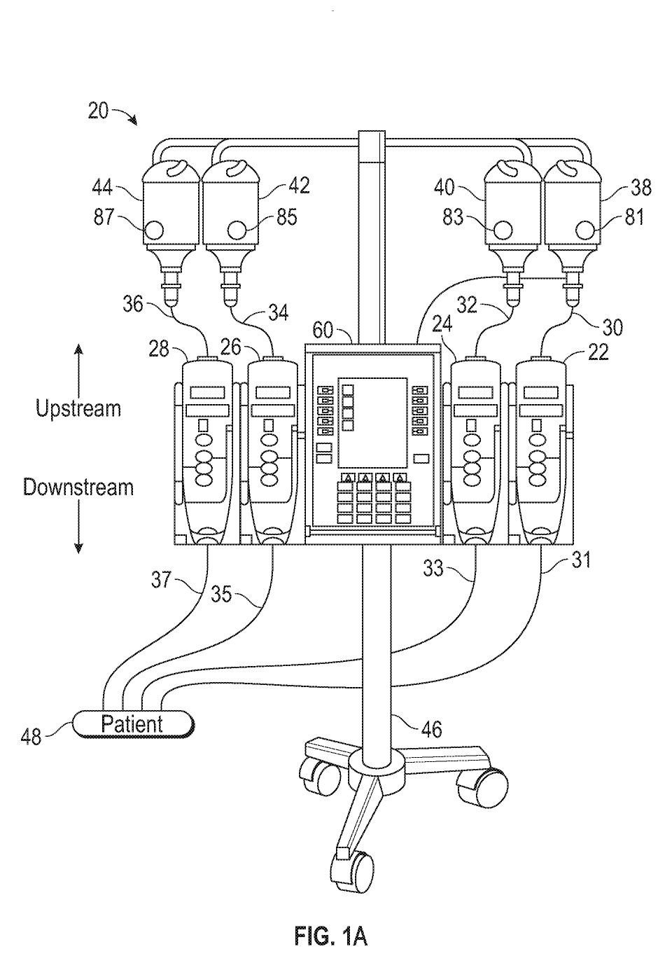

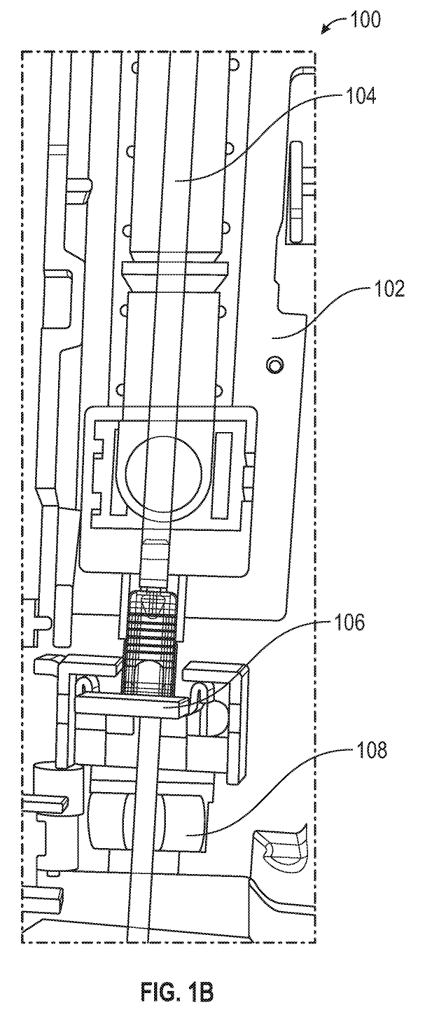

Referring now in more detail to the drawings in which like reference numerals refer to like or corresponding elements among the several views, there is shown in FIG. 1A a patient care system 20 having four infusion pumps 22, 24, 26, and 28 each of which is fluidly connected with an upstream fluid line 30, 32, 34, and 36, respectively. Each of the four infusion pumps 22, 24, 26, and 28 is also fluidly connected with a downstream fluid line 31, 33, 35, and 37, respectively. The fluid lines can be any type of fluid conduit, such as an IV administration set, through which fluid can flow through. It should be appreciated that any of a variety of pump mechanisms can be used including syringe pumps.

Fluid supplies 38, 40, 42, and 44, which may take various forms but in this case are shown as bottles, are inverted and suspended above the pumps. Fluid supplies may also take the form of bags or other types of containers including syringes. Both the patient care system 20 and the fluid supplies 38, 40, 42, and 44 are mounted to a roller stand, IV pole 46, table top, etc.

A separate infusion pump 22, 24, 26, and 28 is used to infuse each of the fluids of the fluid supplies into the patient. The infusion pumps are flow control devices that will act on the respective fluid line to move the fluid from the fluid supply through the fluid line to the patient 48. Because individual pumps are used, each can be individually set to the pumping or operating parameters required for infusing the particular medical fluid from the respective fluid supply into the patient at the particular rate prescribed for that fluid by the physician. Such medical fluids may include drugs or nutrients or other fluids. The infusion pumps 22, 24, 26, and 28 are controlled by a pump control unit 60.

Fluid supplies 38, 40, 42, and 44 are each coupled to an electronic data tag 81, 83, 85, and 87, respectively, or to an electronic transmitter. Any device or component associated with the infusion system may be equipped with an electronic data tag, reader, or transmitter.

Typically, medical fluid administration sets have more parts than are shown in FIG. 1. Many have check valves, drip chambers, valves with injection ports, connectors, and other devices well known to those skilled in the art. These other devices have not been included in the drawings so as to preserve clarity of illustration.

As shown in FIG. 1B, an infusion pump system 100 has a pump 102 (e.g., large volume pump) that receives a pump segment 104. The pump 102 manipulates the tubing of the pump segment 104 to cause fluid to move through and out of the pump segment 104 at a set flow rate. The infusion pump system 100 also includes a flow stop 106 for occluding the pump segment 104 to stop fluid flow, as well as an air-in-line sensor 108 that receives tubing of the pump segment 104 and provides for detection of air bubbles within the fluid and/or the tubing.

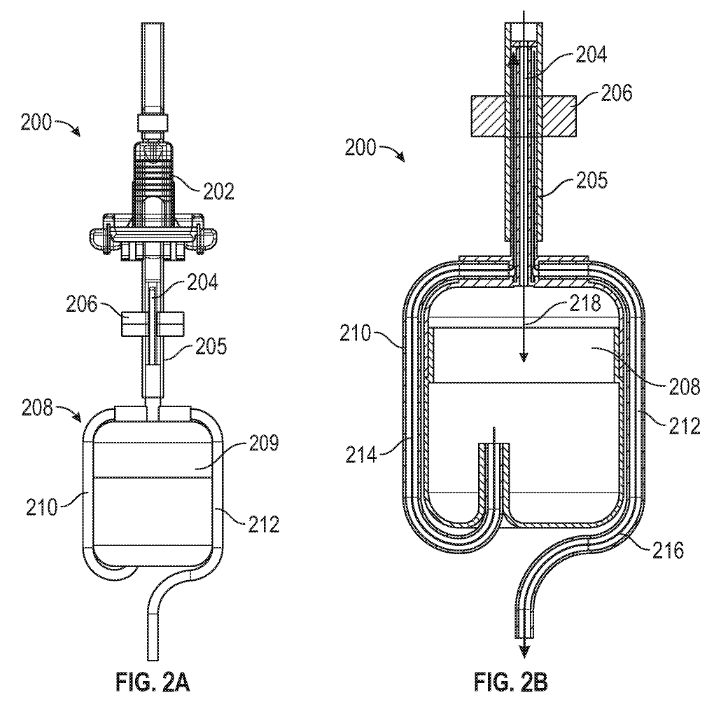

Turning to FIGS. 2A and 2B, an air-in-line system 200 may include a pump segment 202, an air-in-line sensor assembly 206 and an air capturing device 208. The air capturing device 208 includes a container 209, a first side channel 210 and a second side channel 212. A flow cannula 204 is disposed within IV tubing 205 that is received by the air-in-line sensor assembly 206.

In use, the air capture device 208 may separate fluid and air, capture the air and direct fluid flow through the air-in-line sensor assembly 206. As shown in FIG. 2B, the fluid flow from the pump segment 202 passes through the flow cannula 204 disposed within IV tubing 205 and enters the air capture device 208 as shown by flow 218. Air, or any gas, is separated out of the fluid within the container 209 and the resulting fluid flows out of the container 209 through the first side channel 210 and back up into the flow cannula 204 as shown by flow 214. The fluid further flows back down from the flow cannula 204 and through the second side channel 212 as shown by flow 216. The flow 216 may be directed to a downstream IV tube or IV set that may be connected to a patient (not shown).

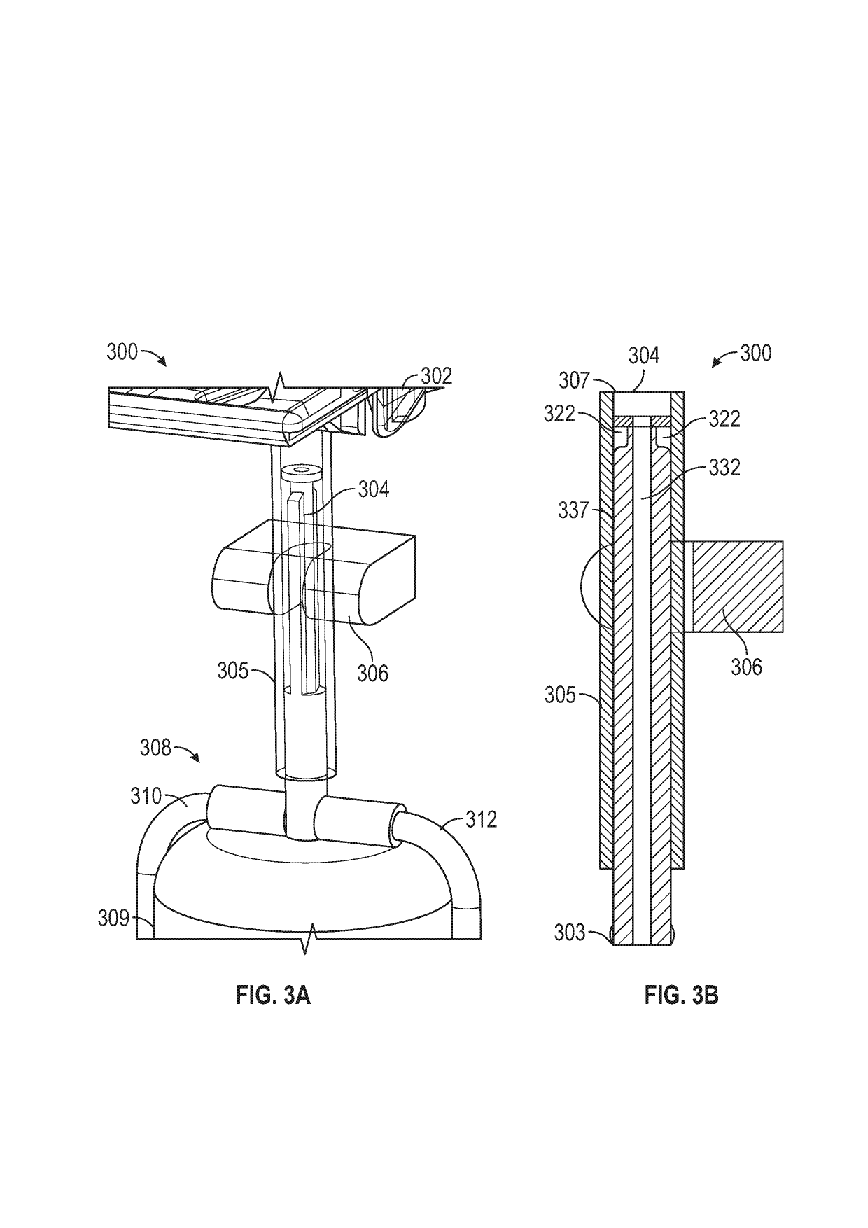

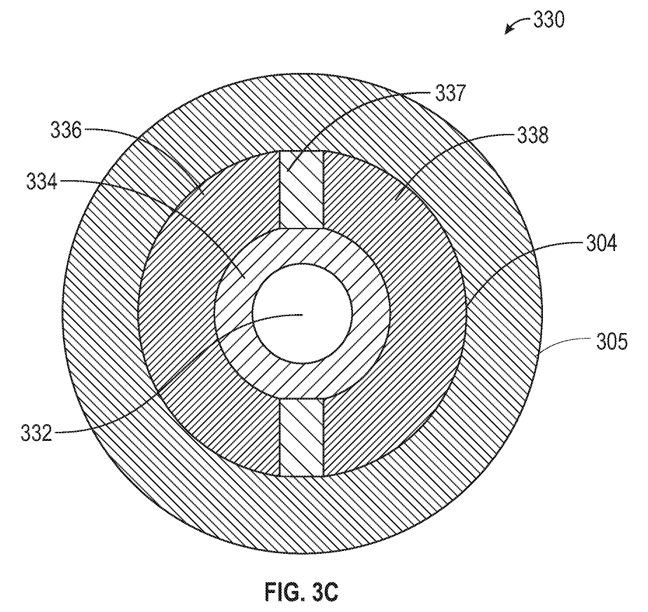

As shown in FIG. 3A, an air-in-line system 300 may include a flow cannula 304 disposed within IV tubing 305 that is disposed within an infusion pump 302, an air-in-line sensor assembly 306 and an air capturing device 308 having a first side channel 310 and a second side channel 312. The flow cannula 304 is disposed within IV tubing 305 that is received within the air-in-line sensor assembly 306. For example, the air-in-line sensor assembly 306 may include a transmitter (e.g., optical, acoustic) positioned on one side of the IV tubing 305 and a receiver (e.g., optical, acoustic) positioned on the other side of the IV tubing 305 so that fluid flowing through the IV tubing 305 (e.g., clear flexible tubing) may be subjected to optical or acoustic waves from the transmitter.

As shown in more detail in FIGS. 3B and 3C, the flow cannula 304 includes a center lumen 332 extending from an inlet end 307 to an outlet end 303 and a center lumen wall 334 encircling a portion of or the entire length of the center lumen 332. For example, the center lumen 332 and center lumen wall 334 may form a tube. A first channel 336 and a second channel 338 are disposed adjacent the center lumen wall 334, the first and second channels 336, 338 being separated by channel walls 337. Flow holes 322 are disposed in the channel walls 337 adjacent the inlet end 307 and provide a fluid pathway between the first channel 336 and the second channel 338.

In use, the air capture device 308 may separate fluid and air, capture the air and direct fluid flow through the air-in-line sensor assembly 306. Here, an upstream fluid flow from the infusion pump 302 passes through the center lumen 332 of the flow cannula 304 disposed within the IV tubing 305 and enters a container 309 of the air capture device 308. Similarly to air-in-line system 200 discussed above, air, or any gas, is separated out of the fluid within the container 309 and the resulting fluid flows out of the container 309 through the first side channel 310 and back up into the first channel 336 of the flow cannula 304. When the fluid reaches the flow holes 322 in the channel walls 337, the fluid flows through the flow holes 322, down the second channel 338 of the flow cannula 304 and into the second side channel 312 of the air capture device 308. The fluid then flows through the second side channel 312 to a downstream IV tube or IV set that may be connected to a patient (not shown).

Here, the initial fluid flow down through the center lumen 332 of the flow cannula 304 is shielded from the air-in-line sensor assembly 306 by the center lumen wall 334, thus preventing the initial fluid flow, which may have air bubbles, this is going into the air capture device 308 from triggering the air-in-line sensor assembly 306 prematurely. The center lumen wall 334 is sufficiently rigid to reflect transmitter signals (e.g., ultrasonic waves) around the center lumen 332 rather than passing through the center lumen 332, thus decreasing the sensitivity of the propagated transmitter signals to the fluid properties within the center lumen 332. Here, the center lumen wall 334 may be formed from a rigid plastic or metal, for example.

The fluid within the first channel 336 and the second channel 338 surrounds the center lumen wall 334 and thus provide the primary medium for the transmitter signals coming from the air-in-line sensor assembly 306. Thus, the fluid containing any air or gas going through the center lumen 332 is shielded from the air-in-line sensor assembly 306, while the fluid having the air or gas removed by the air capture device 308 that is within the first channel 336 and the second channel 338 is available to the transmitter signals coming from the air-in-line sensor assembly 306. Subsequently, the transmitter signal interaction with only the fluid within the first channel 336 and the second channel 338 and not the fluid within the center lumen 332 is subsequently received by the receiver of the air-in-line sensor assembly 306. This reduces or eliminates false positive readings of air/gas bubbles within the initial fluid flow coming from upstream in the infusion pump 302, while providing accurate positive readings of any air/gas bubbles remaining in the fluid after the fluid exits the air capture device 308 back into the flow cannula 304.

In aspects of the disclosure, an air capture device (e.g., air capture device 208, 308) may be provided that captures all air in the fluid from within and above the pump segment (e.g., pump segment 104, 202), thus reducing risk to the patient of air ingress and reducing nuisance air-in-line alarms (e.g., from air-in-line sensor assembly 206, 306) for clinicians. Further, if a failure with the air capture device (e.g., air capture device 208, 308) occurs, the air-in-line sensor (e.g., air-in-line sensor assembly 206, 306) will still be able to sense the air in the fluid going downstream and thus protect the patient as intended.

In aspects of the disclosure, the flow cannula 204, 304 is inserted into the IV tubing 205, 305 and positioned between portions of the air-in-line sensor assembly 206, 306. In aspects of the disclosure, the flow cannula 204, 304 may be integrally formed as a part of the IV tubing 205, 305, such as coextruded as part of the IV tubing 205, 305, for example.

One or more embodiments of the present disclosure are directed to a device for capturing air in a fluid pathway, the device including intravenous (IV) tubing configured to be disposed within an air-in-line sensor and a flow cannula disposed within the IV tubing, the flow cannula comprising a center lumen disposed axially along the flow cannula, wherein the flow cannula is configured to direct an incoming fluid flow from a pump segment through the center lumen and into an air capture device, and wherein the flow cannula is configured to direct processed fluid received back from the air capture device through the flow cannula externally to the center lumen and internally to the IV tubing.

In aspects of the disclosure, the device is configured to be disposed in a fluid line of an infusion pump. In aspects of the disclosure, the flow cannula is inserted into the IV tubing and positioned in between a transmitter and a receiver of the air-in-line sensor. In aspects of the disclosure, the flow cannula is an integrally formed portion of the IV tubing disposed within a fluid flow path of the IV tubing and positioned in between a transmitter and a receiver of the air-in-line sensor. In aspects of the disclosure, the IV tubing and the flow cannula is a co-extruded component.

In aspects of the disclosure, the flow cannula comprises an axially disposed center lumen wall that defines the center lumen. In aspects of the disclosure, the flow cannula comprises one or more channels disposed adjacent an external surface of the center lumen wall. In aspects of the disclosure, the flow cannula comprises a first channel and a second channel disposed axially on opposing sides of the center lumen. In aspects of the disclosure, the flow cannula comprises first and second channel walls extending radially from the center lumen wall and separating the first channel from the second channel. In aspects of the disclosure, the flow cannula comprises a first flow hole in the first channel wall and a second flow hole in the second channel wall, the first and second flow holes disposed at a fluid inlet end of the flow cannula. In aspects of the disclosure, the first channel is configured to couple with a first side channel of the air capture device and the second channel is configured to couple with a second side channel of the air capture device.

In aspects of the disclosure, the flow cannula comprises a total fluid flow path that: directs fluid flow from the fluid inlet end of the flow cannula through the center lumen into the air capture device; directs fluid flow from the first side channel of the air capture device into the first channel of the flow cannula; directs fluid flow from the first channel of the flow cannula, through the first and second flow holes and into the second channel of the flow cannula; and directs fluid flow from the second channel of the flow cannula into the second side channel of the air capture device. In aspects of the disclosure, the center lumen wall is rigid and configured to reflect ultrasonic waves around the center lumen and not pass through the center lumen. In aspects of the disclosure, the center lumen wall comprises one of rigid plastic and metal.

One or more embodiments of the present disclosure are directed to a method for capturing air in a fluid pathway, the method including placing intravenous (IV) tubing comprising a flow cannula within an air-in-line sensor of an infusion pump; positioning the flow cannula between a transmitter and a receiver of the air-in-line sensor; directing fluid flow from the infusion pump through a center lumen of the flow cannula having a center lumen wall configured to reflect waves from the transmitter from passing through the center lumen; directing fluid flow from the center lumen into an air capture device; and directing fluid flow from the air capture device back into the flow cannula to fill one or more channels around the center lumen wall with fluid.

In aspects of the disclosure, the method comprises removing, by the air capture device, air from the fluid received from the center lumen of the flow cannula. In aspects of the disclosure, the method comprises passing waves from the transmitter through the fluid in the one or more channels to a receiver of the air-in-line sensor. In aspects of the disclosure, the method comprises determining, by the air-in-line sensor, whether air remains in the fluid received from the air capture device; and generating, by a processor, an alert if it is determined that air remains in the fluid received from the air capture device.

One or more embodiments of the present disclosure are directed to a flow cannula for use within intravenous (IV) tubing, the flow cannula including an axially disposed center lumen wall defining a center lumen; a first channel wall extending radially from the center lumen wall; a second channel wall extending radially from the center lumen wall and on an opposite side of the center lumen wall from the first channel wall; a first flow channel disposed between a first side of the first channel wall, a first side of the second channel wall and a first portion of the center lumen wall; a second flow channel disposed between a second side of the first channel wall, a second side of the second channel wall and a second portion of the center lumen wall; and a flow hole disposed in one of the first channel wall and the second channel wall, the flow hole fluidly coupling the first flow channel and the second flow channel.

In aspects of the disclosure, the flow cannula is configured to: direct fluid flow from a fluid inlet end of the flow cannula through the center lumen and into an inlet of an air capture device; direct fluid flow exiting an outlet of the air capture device into the first flow channel, the first flow channel bounded by a first internal surface of the IV tubing; direct fluid flow from the first flow channel through the flow hole and into the second flow channel, the second flow channel bounded by a second internal surface of the IV tubing; and direct fluid flow from the second flow channel out of the flow cannula.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but is to be accorded the full scope consistent with the language claims and to encompass all legal equivalents. Notwithstanding, none of the claims are intended to embrace subject matter that fails to satisfy the requirement of 35 U.S.C. §101, 102, or 103, nor should they be interpreted in such a way.

What is claimed is:

1. A device for capturing air in a fluid pathway, the device comprising:
intravenous (IV) tubing configured to be disposed within an air-in-line sensor; and
a flow cannula disposed within the IV tubing, the flow cannula comprising a center lumen disposed axially along the flow cannula,
wherein the flow cannula is configured to direct an incoming fluid flow from a pump segment through the center lumen and into an air capture device,
and wherein the flow cannula is configured to direct processed fluid received back from the air capture device through the flow cannula externally to the center lumen and internally to the IV tubing.

2. The device of claim 1, wherein the device is configured to be disposed in a fluid line of an infusion pump.

3. The device of claim 1, wherein the flow cannula is inserted into the IV tubing and positioned in between a transmitter and a receiver of the air-in-line sensor.

4. The device of claim 1, wherein the flow cannula is an integrally formed portion of the IV tubing disposed within a fluid flow path of the IV tubing and positioned in between a transmitter and a receiver of the air-in-line sensor.

5. The device of claim 4, wherein the IV tubing and the flow cannula is a co-extruded component.

6. The device of claim 1, wherein the flow cannula comprises an axially disposed center lumen wall that defines the center lumen.

7. The device of claim 6, wherein the flow cannula comprises one or more channels disposed adjacent an external surface of the center lumen wall.

8. The device of claim 7, wherein the flow cannula comprises a first channel and a second channel disposed axially on opposing sides of the center lumen.

9. The device of claim 8, wherein the flow cannula comprises first and second channel walls extending radially from the center lumen wall and separating the first channel from the second channel.

10. The device of claim 9, wherein the flow cannula comprises a first flow hole in the first channel wall and a second flow hole in the second channel wall, the first and second flow holes disposed at a fluid inlet end of the flow cannula.

11. The device of claim 10, wherein the first channel is configured to couple with a first side channel of the air capture device and the second channel is configured to couple with a second side channel of the air capture device.

12. The device of claim 11, wherein the flow cannula comprises a total fluid flow path that:
directs fluid flow from the fluid inlet end of the flow cannula through the center lumen into the air capture device;
directs fluid flow from the first side channel of the air capture device into the first channel of the flow cannula;
directs fluid flow from the first channel of the flow cannula, through the first and second flow holes and into the second channel of the flow cannula; and
directs fluid flow from the second channel of the flow cannula into the second side channel of the air capture device.

13. The device of claim 6, wherein the center lumen wall is rigid and configured to reflect ultrasonic waves around the center lumen and not pass through the center lumen.

14. The device of claim 13, wherein the center lumen wall comprises one of rigid plastic and metal.

15. A method for capturing air in a fluid pathway, the method comprising:
placing intravenous (IV) tubing comprising a flow cannula within an air-in-line sensor of an infusion pump;
positioning the flow cannula between a transmitter and a receiver of the air-in-line sensor;
directing fluid flow from the infusion pump through a center lumen of the flow cannula having a center lumen wall configured to reflect waves from the transmitter from passing through the center lumen;
directing fluid flow from the center lumen into an air capture device; and
directing fluid flow from the air capture device back into the flow cannula to fill one or more channels around the center lumen wall with fluid.

16. The method of claim 15, comprising:
removing, by the air capture device, air from the fluid received from the center lumen of the flow cannula.

17. The method of claim 16, comprising:
passing waves from the transmitter through the fluid in the one or more channels to a receiver of the air-in-line sensor.

18. The method of claim 17, comprising:
determining, by the air-in-line sensor, whether air remains in the fluid received from the air capture device; and
generating, by a processor, an alert if it is determined that air remains in the fluid received from the air capture device.

19. A flow cannula for use within intravenous (IV) tubing, the flow cannula comprising:
an axially disposed center lumen wall defining a center lumen;
a first channel wall extending radially from the center lumen wall;
a second channel wall extending radially from the center lumen wall and on an opposite side of the center lumen wall from the first channel wall;
a first flow channel disposed between a first side of the first channel wall, a first side of the second channel wall and a first portion of the center lumen wall;
a second flow channel disposed between a second side of the first channel wall, a second side of the second channel wall and a second portion of the center lumen wall; and
a flow hole disposed in one of the first channel wall and the second channel wall, the flow hole fluidly coupling the first flow channel and the second flow channel.

20. The flow cannula of claim 19, wherein the flow cannula is configured to:
direct fluid flow from a fluid inlet end of the flow cannula through the center lumen and into an inlet of an air capture device;
direct fluid flow exiting an outlet of the air capture device into the first flow channel, the first flow channel bounded by a first internal surface of the IV tubing;
direct fluid flow from the first flow channel through the flow hole and into the second flow channel, the second flow channel bounded by a second internal surface of the IV tubing; and
direct fluid flow from the second flow channel out of the flow cannula.
